(19) United States
(12) Patent Application Publication
(10) Pub. No.: US 2012/0115772 A1
(43) Pub. Date: May 10, 2012

(54) CONJUGATION METHOD

(76) Inventors: Ji-Won Choi, London (GB); Antony Godwin, London (GB); Estera Pawlisz, London (GB)

(21) Appl. No.: 13/384,401

(22) PCT Filed: Jul. 15, 2010
(86) PCT No.: PCT/GB2010/001356

§ 371 Date: Jan. 17, 2012

(30) Foreign Application Priority Data
Jul. 17, 2009 (GB) 0912485.0

(51) Int. Cl. A61K 38/02; A61P 29/00; A61P 25/00; C07K 17/06

(52) U.S. Cl. 514/1.1; 530/351

(57) ABSTRACT

A process for the conjugation of a polymer to a protein, which comprises reacting a polymeric conjugating agent with said protein, in an aqueous medium, in the presence of an amphipathic sugar polymer. The process is particularly useful when conjugating PEG to proteins, particularly to proteins which have previously proved difficult to PEGylate in acceptable yields, for example INF-β.

This invention relates to an improved method of conjugating proteins to polymers, particularly polyethylene glycol (PEG).

Many proteins do not possess the properties required to achieve efficacy in clinical medical use. For example, many native proteins do not make good medicines because upon administration to patients there are several inherent drawbacks that include: (1) proteins are digested by many endo- and exo-peptidases present in blood or tissue; (2) many proteins are immunogenic to some extent; and (3) proteins can be rapidly excreted by kidney ultrafiltration and by endocytosis. Some molecules which might find utility as active therapeutic agents in medicines are systemically toxic or lack optimal bioavailability and pharmacokinetics. When proteins clear from the blood circulation quickly they typically have to be administered to the patient frequently. Frequent administration further increases the risk of toxicity, especially immunologically derived toxicities.

Water soluble, synthetic polymers, particularly polyalkylene glycols, are widely used to conjugate therapeutically active molecules such as proteins, peptides and low molecular weight drugs. These therapeutic conjugates have been shown to alter pharmacokinetics favourably by prolonging circulation time and decreasing clearance rates, decreasing systemic toxicity, and in several cases, displaying increased clinical efficacy. The process of covalently conjugating polyethylene glycol, PEG, to proteins is commonly known as “PEGylation”. PEGylation is a clinically proven strategy to improve the efficacy of protein-based medicines. It is also an enabling technology that can address developmental issues such as poor pharmacokinetic, immunogenic and physicochemical properties. It is, however, a technology which may be difficult to apply in practice, and many proteins are difficult to PEGylate successfully.

Interferons (IFNs) are proteins that belong to the class of cytokines. Their main functions are protection of cells from viral infection as well as initiation of antiproliferative response against malignant cells. IFN-β is used as a medicine to treat people with multiple sclerosis.

It is available in two forms, the mammalian-expressed and glycosylated IFN-β-1a and the E. coli-expressed IFN-β-1b. Both forms are structurally similar but differ in the number of cysteine residues. IFN-β-1a possesses three cysteines and IFN-β-1b has two cysteines that form a single disulfide bridge. Although the E. coli-expressed protein is more cost-effective, it is intrinsically less stable than its mammalian counterpart due to the absence of glycosylation, and it is particularly prone to aggregation. Both IFN-β-1a and IFN-β-1b have relatively short half-lives due to their small size (about 19 kDa) leading to frequent dosing for MS sufferers.

Various attempts to PEGylate INF-β have been reported, for example Pepinsky et al., J. Pharmacol. Exp. Ther. 2001, 297, 1059-1066, and Basu et al., Bioconjugate Chem. 2006, 17, 618-630. Although these PEGylation methods have been shown to improve INF-β stability, the synthetic methods are generally of very low efficiency.

EP 1,666,496A1 claims a method for producing an interferon-β complex, comprising binding interferon-β to polyethylene glycol in the presence of at least one additive selected from the group consisting of oligosaccharides having 5 or less sugar units, monosaccharides, their corresponding sugar alcohols, and C₂₋₆ polyhydric alcohols. WO 2005/055946 describes conjugates between Granulocyte Colony Stimulating Factor (G-CSF) and an enzymatically transferable saccharyl moiety that includes a poly(ethylene glycol) moiety within its structure. WO 2006/127910 describes conjugates in which erythropoietin (EPO) is conjugated through a glycosyl linking group to a polymeric modifying moiety, for example PEG.

WO 2005/007197 describes an improved method for the conjugation of polymers, particularly PEG, to proteins containing a disulfide bond. In this method, the disulfide bond is reduced to produce two free cysteine residues, and the polymer is conjugated to both of these residues. The PEGylation of INF-α is described, and because INF-β contains a disulfide bond, the PEGylation of INF-β can also be carried out using this method. However, yields tend to be relatively low because the PEGylation can only proceed efficiently with the monomeric protein: INF-β tends to aggregate in aqueous media and, crucially, increased aggregation occurs on reduction of the disulfide bond.

WO 2005/103067 describes a method of stabilising a protein, which comprises contacting the protein with an amphipathic sugar polymer. The method finds utility in aspects of protein processing which involve protein folding, purification, concentration and/or storage. WO 2005/026196 and WO 2005/103068 describe similar technology. Such amphipathic sugar polymers would not be expected to find utility in chemical processes such as those for the conjugation of a polymer to a protein, because hydrophobic interaction between the protein and the amphipathic sugar polymer would be expected to block suitable sites of conjugation between the protein and the polymer. We have now found, surprisingly, that the presence of such amphipathic sugar polymers can enhance the yields of conjugated product when chemically conjugating a polymer to a protein. This new method is particularly useful when conjugating PEG to proteins, including proteins which have previously proved difficult to PEGylate in acceptable yields, for example INF-β.

The present invention therefore provides a process for the conjugation of a polymer to a protein, which comprises reacting a polymeric conjugating agent with said protein, in an aqueous medium, in the presence of an amphipathic sugar polymer.

The term “amphiphilic sugar polymer” as used herein encompasses polymeric and oligomeric saccharide molecules comprising 3 or more, preferably 6 or more, especially 10 or more, monosaccharide units, and may be linear or non-linear. The sugar polymer may comprise one or more sugar(s) selected from the group consisting of: erythrose, threose, ribose, arabinose, xylose, lyxose, allose, altrose, glucose, mannose, gulose, idose, galactose, talose, psicose, fructose, sorbose, tagatose, xylulose and ribulose. Of these sugars, glucose, fructose, mannose and/or galactose, are the four most common simple (monomer) sugar units. The sugar polymer derivative may for example be a fructosan or a glucosan derivative. It may for example be a dextran, cellulose, amylose, starch, pullulan, mannan, chitin, chitosan, inulin, levan, xylan, cyclodextrin, cycloamylose or a derivative thereof. Suitable sugar polymers are disclosed in U.S. Pat. Nos. 5,202,433, 5,204,457, WO 2005/103068, WO 2005/026196 and WO 2005/103067.

Amphipathic sugar polymers contain non-polar, hydrophobic substituents in addition to sugar groups. Suitable substituents may be selected from the group consisting of alkyl groups having from 1 to 25 carbon atoms, alkenyl and alkynyl groups having from 2 to 25 carbon atoms, haloalkyl groups having from 1 to 25 carbon atoms, cycloalkyl groups having from 3 to 9 carbon atoms, aryl groups having from 6 to 14 carbon atoms, aralkyl groups comprising alkyl groups having from 1 to 25 carbon atoms which are substituted with 1 or more aryl groups having from 6 to 14 carbon atoms, fatty acid groups having from 2 to 25 carbon atoms and polyols having from 1 to 25 carbon atoms. One or more substituents per carbohydrate molecule can be present. The alkyl, alkenyl, alkynyl, haloalkyl, aralkyl, fatty acid and polyol groups may be straight chain or branched chain groups. Preferably, the substituents are selected from alkyl, alkenyl and alkynyl groups having from 2 to 25 carbon atoms, more preferably they are selected from alkyl, alkenyl and alkynyl groups having from 3 to 22 carbon atoms, and most preferably they are selected from alkyl, alkenyl and alkynyl groups having from 3 to 18 carbon atoms.

The sugar polymer can be a cyclic sugar polymer derivative comprising 3 or more, especially 6 or more, particularly 10 or more, saccharide units, which may be a glucosan, such as a cyclodextrin derivative.

The sugar polymer is preferably a linear or branched sugar polymer comprising three or more monosaccharide units, such as a fructosan, e.g. an inulin derivative, or a glucoside such as a glucoside hydrocarbyl derivative. Preferred sugar polymers include inulin derivatives of formula (IV) and glucoside hydrocarbyl derivatives of formula (V) described below, particularly inulin derivatives. A glucoside hydrocarbyl derivative of formula (V) is preferably a hydrocarbyl urethane of a starch hydrolysate, particularly preferably a glucoside hydrocarbyl derivative composed of units of formula (VI) defined below.

Suitably an inulin derivative or glucoside hydrocarbyl derivative has a degree of polymerisation of from about 3 to 500, 3 to 250 or 3 to 100, preferably from 3 to 50, more preferably from 10 to 50, yet more preferably from 15 to 40, further preferably from 20 to 30, e.g. 20, 21, 22, 23, 24, 25, 26, 27, 28, 29, 30.

Sugar polymers suitable for the method described herein typically have an average degree of substitution by hydrophobic, non-polar substituents per saccharide unit of from 0.01 to 3.0 or 0.02 to 3.0, preferably from 0.05 to 1.0, most preferably from 0.05 to 0.5. Preferred inulin derivatives typically have an average degree of substitution per saccharide unit in the range of from 0.01 to 3.0, or 0.02 to 3.0, preferably from 0.02 to 1.0 or 0.05 to 1.0, most preferably from 0.05 to 0.5 or 0.03 to 0.3. Preferred glucoside hydrocarbyl derivatives generally have a degree of substitution in the range of from 0.01 to 2.0, preferably 0.02 to 1.0, more preferably 0.03 to 0.3.

Various inulin derivatives and methods for preparing them are described in U.S. Pat. No. 6,534,647. Inulins derivatised with hydrophobic alkyl chains on the polyfructose backbone are commercially available. A particularly suitable inulin derivative is Inutec® SP1 (Orafti, Belgium).

A preferred inulin derivative is a compound of formula (IV):

G(O—CO—NH—R¹)ₐ—[F(O—CO—NH—R²)_{b}]ₙ (IV)

wherein:
G is a terminal glucosyl unit in which one or more hydroxyl groups thereof may be substituted with a group or groups of formula (O—CO—NH—R¹);
R¹ is a hydrocarbyl group selected from the group consisting of alkyl groups, alkenyl groups, alkynyl groups, haloalkyl groups, cycloalkyl groups, aryl groups and aralkyl groups and, where there is more than one (O—CO—NH—R¹) group on the glucosyl unit, each R¹ group may be the same or different;
a is an integer of from 0 to 4;
F is a fructosyl unit in which one or more hydroxyl groups thereof may be substituted with a group or groups of formula (O—CO—NH—R²);
R² is a hydrocarbyl group selected from the group consisting of alkyl groups, alkenyl groups, alkynyl groups, haloalkyl groups, cycloalkyl groups, aryl groups and aralkyl groups and, where there is more than one (O—CO—NH—R²) group on the fructosyl unit, each R² group may be the same or different; b is an integer of from 0 to 3 and from 0 to 4 for the terminal fructosyl unit;
n is an integer of from 2 to 499 preferably of from 2 to 249, 2 to 99, 2 to 49, 9 to 49, 14 to 39, 19 to 29, or 19 to 24,
each unit of formula F(O—CO—NH—R²)_{b} may be the same or different from any other unit of formula F(O—CO—NH—R²)_{b}; and
the average degree of substitution per glucosyl or fructosyl unit is from 0.01 to 3.0.

Where R¹ or R² is an alkyl group, it is suitably a linear or branched chain alkyl group having from 1 to 25 carbon atoms; preferably, it has from 3 to 22 carbon atoms, and most preferably from 3 to 18 carbon atoms.

Where R¹ or R² is an alkenyl group, it is suitably a linear or branched chain alkenyl group having from 2 to 25 carbon atoms; preferably, it has from 3 to 22 carbon atoms, and most preferably from 3 to 18 carbon atoms.

Where R¹ or R² is an alkynyl group, it is suitably a linear or branched chain alkynyl group having from 2 to 25 carbon atoms; preferably, it has from 3 to 22 carbon atoms, and most preferably from 3 to 18 carbon atoms.

Where R¹ or R² is a haloalkyl group, it is suitably an alkyl group as defined above which is substituted with 1 or more halogen atoms (e.g. fluorine, chlorine or bromine atoms). Preferably, said haloalkyl groups have from 1 to 3 halogen atom substituents, more preferably from 1 to 3 fluorine or chlorine atom substituents. Preferably, said haloalkyl groups have from 3 to 22 carbon atoms, and most preferably from 3 to 18 carbon atoms.

Where R¹ or R² is a cycloalkyl group, it is suitably a cyclic alkyl group having from 3 to 9 carbon atoms; preferably, said cycloalkyl groups have from 3 to 7 carbon atoms and most preferably from 4 to 6 carbon atoms.

Where R¹ or R² is an aryl group, it is suitably an aromatic group having from 6 to 14 carbon atoms in one or more rings, e.g. a phenyl group or a naphthyl group.

Where R¹ or R² is an aralkyl group, it is suitably an alkyl group as defined above that is substituted with one or more aromatic groups having from. 6 to 14 carbon atoms in one or more rings, e.g. a benzyl group or a triphenylmethyl group.

Each group R¹ and R² may be selected from alkyl groups having from 1 to 25 carbon atoms, and alkenyl and alkynyl groups having from 2 to 25, preferably 3 to 22, most preferably 3 to 18 carbon atoms. One or more of the groups R¹ and R² can be an alkyl group having from 1 to 25, preferably 3 to 22, most preferably 3 to 18 carbon atoms; suitably one or more of groups R¹ and R² is an alkenyl or alkynyl group having from 2 to 25, preferably 3 to 22, most preferably 3 to 18 carbon atoms. Each alkyl group R¹ and R² can be a linear alkyl group having from 1 to 25, preferably 3 to 22, most preferably 3 to 18 carbons or branched alkyl group having from 3 to 25, preferably 3 to 22, most preferably 3 to 18 carbons.

The average degree of substitution per glucosyl or fructosyl unit is suitably from in the range of from 0.01 to 3.0, or 0.02 to 3.0, preferably from 0.02 to 1.0 or 0.05 to 1.0, most preferably from 0.05 to 0.5 or 0.03 to 0.3. The compound of formula (IV) can be a polydisperse linear or slightly branched inulin N-alkylurethane, e.g. selected from the group consisting of inulin N-n-octyl-carbamates, inulin N-n-dodecylcarbamates and inulin N-n-octadecylcarbamates.

The sugar polymer may be a glucoside hydrocarbyl derivative and is further preferably a glucoside hydrocarbyl derivative of formula (V):

[G(O—CO—NH—R¹)ₐ]ₙ (V)

wherein:
G is a glucosyl unit in which one or more hydroxyl groups thereof may be substituted with a group or groups of formula (O—CO—NH—R¹), in which R¹ has the meaning given above;
a is an integer of from 0 to 4 for a terminal glucosyl unit, 0 to 3 for a non-branched, non-terminal glucosyl unit and 0 to 2 for a branched, non-terminal glucosyl unit;
n is an integer of from 3 to 499 preferably of from 3 to 249, 3 to 99, 3 to 49, 9 to 49, 14 to 39, 19 to 29, or 19 to 24,
each unit of formula G(O—CO—NH—R¹)ₐ may be the same or different from any other unit of formula G(O—CO—NH—R¹)ₐ; and
the average degree of substitution per glucosyl unit is from 0.01 to 2.0.

In a compound of formula (V), the average degree of substitution per glucosyl unit is from 0.01 to 2.0, preferably from 0.02 to 1.0, and most preferably from 0.03 to 0.3.

The compound of formula (V) can be a polydisperse linear or branched glucoside N-hydrocarbyl urethane. Each glucosyl unit G may be a D-glucosyl or L-glucosyl unit, preferably a D-glucosyl unit. The glucosyl units can be linked via 1,4-linkages or 1,6-linkages, and each linkage can be an α-linkage or a β-linkage.

Said compounds of formula (V) are preferably hydrocarbyl urethanes of starch hydrolysates.

Particularly preferred hydrocarbyl urethanes of starch hydrolysates are glucoside hydrocarbyl derivatives composed of units of formula (VI):

G′(O—CO—NH—R¹)_{b} (VI)

wherein
G′ represents a glucosyl unit of a starch hydrolysate molecule, the starch hydrolysate having a Dextrose Equivalent (D. E.) ranging from 1 to 47,
R¹ is a hydrocarbyl group as defined above, and
b represents the number of hydrocarbyl carbamate groups per glucosyl unit, which number is commonly expressed as the degree of substitution (DS), i.e. the average number of hydrocarbyl substituents per glucosyl unit of the glucosyl hydrocarbyl urethane of formula (V), with said DS value ranging from 0.01 to 2.0.

The number of hydroxyl groups per glucosyl unit of the subject glucoside molecules which theoretically can be substituted by a carbamate group is, for a non-terminal, non-branched glucosyl unit, a maximum of 3, whereas the number for a terminal or for a non-terminal branched glucosyl unit is, respectively, 4 or 2. Furthermore, since the DS represents an average number of substituents per glucosyl unit, it is evident that in a glucoside N-hydrocarbyl carbamate molecule there may be glucosyl units present which are not substituted by a hydrocarbyl carbamate group (thus b in formula (VI) being zero for said glucosyl unit).

Starch hydrolysates commonly appear in the form of a polydisperse mixture of glucoside molecules. Accordingly, when such a mixture is used, as is usually the case, as starting material for the preparation of a glucoside hydrocarbyl urethane, the product obtained is also a corresponding polydisperse mixture of glucoside hydrocarbyl urethanes. Such polydisperse mixtures constitute a preferred embodiment of the glucoside hydrocarbyl urethanes for utilisation in the methods and uses of the present invention.

Commercial grades of starch hydrolysates, composed of said polydisperse mixture of glucoside molecules and having a D. E. ranging from 1 to 47 are very suitable for the preparation of glucoside hydrocarbyl urethanes.

Typically suitable starch hydrolysates for use in the preparation of glucoside N-hydrocarbyl urethanes are for example GLUCIDEX® maltodextrins and GLUCIDEX® dried glucose syrups which are available from ROQUETTE company, such as the maltodextrins of type 1 (potato based with D. E. max 5), type 2 (Waxy Maize based with D. E. max 5), type 6 (Waxy Maize based with D. E. 5 to 8), type 9 (Potato based with D. E. 8 to 10), and maltodextrins of type 12 (D. E. 11 to 14), type 17 (D. E. 15 to 18) and type 19 (D. E. 18 to 20), as well as dried glucose syrups of type 21 (D. E. 20 to 23), type 28E (D. E. 28 to 31), type 29 (D. E. 28 to 31), type 32 (D. E. 31 to 34), type 33 (D. E. 31 to 34), type 38 (D. E. 36 to 40), type 39 (D. E. 38 to 41), type 40 (D. E. 38 to 42) and type 47 (D. E. 43 to 47).

The hydrocarbyl group of the hydrocarbyl urethanes, i.e. the R¹ group in formula (VI) defined herein above, is preferably a saturated C₃-C₂₂ alkyl group, more preferably a saturated C₄-C₁₈ alkyl group, even more preferably a saturated linear C₄-C₁₈ alkyl group, most preferably a saturated linear C₆-C₁₈ alkyl group. Typically suitable alkyl groups include butyl, hexyl, octyl, decyl, dodecyl, tetradecyl, hexadecyl and octadecyl groups.

In the urethane discussed above, all R¹ groups of the units of formula (V) may be the same or different. The latter urethanes can be easily prepared, according to the method described below, by reacting a starch hydrolysate with an isocyanate of formula R¹—NCO which is in fact a mixture of two or more isocyanates bearing different R¹ groups defined above.

Glycoside hydrocarbyl urethanes suitably a degree of substitution (DS) per glucosyl unit of formula (VI) ranging from 0.01 to 2.0, preferably from 0.02 to 1.0, and most preferably from 0.03 to 0.3.

The hydrocarbyl carbamate substituent or substituents can be located at various positions on the glucosyl units of the glucoside hydrocarbyl urethanes.

The use of sugar polymers derived from inulin is preferred. Particularly preferred sugar polymers for use in the process of the present invention are those in the NVoy (Trade Mark) range available from Expedeon Limited, for example NV-10, NV-20 or NV-30 (Trade Marks). Particularly preferred is NV-10 (Trade Mark), an uncharged polyfructose derivative of molecular weight about 5 kDa. This material has a linear carbohydrate backbone, and hydrophobic modifications.

The process of the invention is carried out in an aqueous medium. The solvent may consist of water, or co-solvents and/or solubilising agents may additionally be present. The polymeric conjugation reagent should be one which has at least some solubility in the chosen aqueous reaction medium, but subject to this requirement, any suitable conjugation reagent may be used. These include known reagents which are capable of acylation or alkylation. Such reagents may be mono or multifunctional and include for example the compounds of formulae (I), (II) and (III) defined below; reagents available under the Trade Mark “PermaLink” from Glythera; compounds of the formula

X-[Q-W—(CH═CH)ₚ—(CH₂)₂-L]_{q}

in which X represents a polymer, Q represents a linking group, W represents an electron-withdrawing group, p represents 0 or an integer of from 1 to 4, L represents a leaving group, and q represent an integer of from 1 to 8; PEG carbonates (e.g. PEG-p-nitrophenyl carbonate, PEG-succinimidyl carbonate, PEG-benzotriazolyl carbonate); PEG carboxylates and PEG esters (e.g. PEG-succinimidyl ester and PEG-p-nitrophenyl ester and their derivatives); PEG aldehydes; PEG-tresyl or -tosyl; PEG-dichlortriazine or -chlorotriazine; PEG vinyl sulfone; PEG maleiimide; and PEG-iodoacetamide; and corresponding reagents containing polymers other than PEG. The reagent may be one which conjugates to thiol groups or amine groups, for example lysine or histidine groups, in the protein, but preferably the conjugation reagent is one which conjugates to thiol groups. Any thiol conjugation reagent may be used, for example compounds of the formula (I), (II) and (III) defined below, maleiimides, the “PermaLink” (Trade Mark) reagents, or compounds of the formula X-[Q-W—(CH═CH)ₚ—(CH₂)₂-L]_{q} defined above.

The process of the invention is particularly useful for the conjugation of polymer to proteins which are of low solubility or which tend to aggregate in an aqueous medium, or proteins which become of lower solubility or which show an increased tendency to aggregate during the course of a conjugation reaction in an aqueous medium. The invention is also useful for the conjugation of proteins which tend to be “sticky”, for example certain antibodies or antibody fragments. The process of the invention is particularly useful for the conjugation of polymer to INF-β.

In a preferred embodiment of the process of the invention, the protein to be conjugated is one which contains a disulfide bond. In this case, the conjugation reaction takes place as a two-step process: the protein is mixed with the sugar polymer in an aqueous medium and the disulfide bond is reduced; and the conjugation reagent is then reacted with the protein via the thiol groups produced by reduction of the disulfide bond. Thus, in a preferred embodiment, the invention provides a process for the conjugation of a polymer to a protein containing a disulfide bond, which comprises reducing said disulfide bond in an aqueous medium in the presence of an amphipathic sugar polymer, and subsequently reacting the reduced product with a polymeric conjugating agent.

This preferred embodiment of the invention finds particular utility in the conjugation of polymers to proteins containing a disulfide bond, and in which reduction of the disulfide bond in aqueous medium causes the protein to become less soluble in that medium, and/or to show an increased tendency to aggregate. Typical proteins include Factor Vila, Factor VIII, Factor IX, EPO, G-CSF, or, especially, INF-β. The presence of the sugar polymer has been found to increase yields of the desired product substantially.

The process may be carried out by reducing the disulfide bond in the protein in situ following which the reduced product reacts with the conjugation reagent, for example one of the reagents (I), (II) or (III) below. Preferably the disulfide bond is reduced and any excess reducing agent is removed, for example by buffer exchange, before the conjugation reagent is introduced. The disulfide can be reduced, for example, with dithiothreitol, mercaptoethanol, or tris-carboxyethylphosphine using conventional methods. The process is carried out in an aqueous reaction medium, which may be buffered. The optimum pH for the reaction is generally between about 5.5 and about 8, for example about 7.4, preferably about 6.0-6.5. Reaction temperatures between 3-37° C. are generally suitable.

Depending upon the conjugation reagent used, the protein can be effectively conjugated using a stoichiometric equivalent or a slight excess of conjugation reagent. However, it is also possible to conduct the conjugation reaction with an excess stoichiometry of conjugation reagent, and this may be essential for some reagents. The excess reagent can easily be removed by ion exchange chromatography during subsequent purification.

The sugar polymer should be present at least during the step of reduction of the protein, but it is also preferably present during additional steps in the conjugation process, for example during dissolution or suspension of the protein, addition of the reducing agent, removal of the reducing agent, and addition of the conjugation reagent.

In an especially preferred embodiment, the polymeric conjugation reagent is as described in WO 2005/007197. These reagents find particular utility for conjugation across two thiol groups formed by reduction of a disulfide bond in a protein.

Thus, in a preferred embodiment, the present invention provides a process for the conjugation of a polymer to a protein containing a disulfide bond, which comprises reducing said disulfide bond in an aqueous medium in the presence of an amphipathic sugar polymer, and subsequently reacting the reduced product with either (i) a compound of the general formula

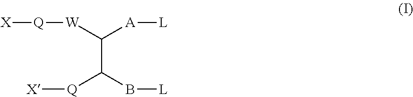

in which one of X and X′ represents a polymer and the other represents a hydrogen atom;
Q represents a linking group;
W represents an electron-withdrawing group, for example a keto group, an ester group —O—CO— or a sulfone group —SO₂—; or, if X′ represents a polymer, X-Q-W together may represent an electron withdrawing group;
A represents a C₁₋₅ alkylene or alkenylene chain;
B represents a bond or a C₁₋₄ alkylene or alkenylene chain; and
each L independently represents a leaving group;
   or (ii) a compound of the general formula

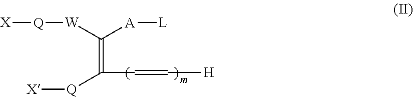

in which X, X′, Q, W, A and L have the meanings given for the general formula I, and in addition if X represents a polymer, X′ and electron-withdrawing group W together with the interjacent atoms may form a ring, and m represents an integer 1 to 4; or (iii) a compound of the general formula

X-Q-W—CR³═C4²-L (III)

in which X, Q and W have the meanings given for the general formula I, and either R³ represents a hydrogen atom or a C₁₋₄alkyl group and L represents a leaving group, or R³ and L together represent a bond; and R⁴ represents a hydrogen atom or a C₁₋₄ alkyl group.

The immediate product of the process described above is a conjugate of the formula

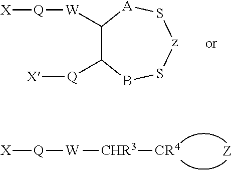

in which W is an electron-withdrawing group and Z is the protein. However, the process of the invention is reversible under suitable conditions. This may be desirable for some applications, for example where rapid release of the protein is required, but for other applications, release of the protein may be undesirable. It may therefore be desirable to stabilise compounds of the formulae above by reduction of the electron-withdrawing moiety W to give a moiety which prevents release of the protein. Accordingly, the process described above may comprise an additional optional step of reducing the electron withdrawing group W in the conjugate. The use of a borohydride, for example sodium borohydride, sodium cyanoborohydride, potassium borohydride or sodium triacetoxyborohydride, as reducing agent is particularly preferred. Other reducing agents which may be used include for example tin(II) chloride, alkoxides such as aluminium alkoxide, and lithium aluminium hydride.

Thus, for example, a moiety W containing a keto group may be reduced to a moiety containing a CH(OH) group; an ether group CH.OR may be obtained by the reaction of a hydroxy group with an etherifying agent; an ester group CH.O.C(O)R may be obtained by the reaction of a hydroxy group with an acylating agent; an amine group CH.NH₂, CH.NHR or CH.NR₂ may be prepared from a ketone by reductive amination; or an amide CH.NHC(O)R or CH.N(C(O)R)₂ may be formed by acylation of an amine. A sulfone may be reduced to a sulfoxide, sulfide or thiol ether. A group X-Q-W— which is a cyano group may be reduced to an amine group.

A key feature of using polymeric conjugation reagents of the formulae I, II and III is that an α-methylene leaving group and a double bond are cross-conjugated with an electron withdrawing function that serves as a Michael activating moiety. If the leaving group is prone to elimination in the cross-functional reagent rather than to direct displacement and the electron-withdrawing group is a suitable activating moiety for the Michael reaction then sequential intramolecular bis-alkylation can occur by consecutive Michael and retro Michael reactions. The leaving moiety serves to mask a latent conjugated double bond that is not exposed until after the first alkylation has occurred and bis-alkylation results from sequential and interactive Michael and retro-Michael reactions as described in J. Am. Chem. Soc. 1979, 101, 3098-3110 and J. Am. Chem. Soc. 1988, 110, 5211-5212.). The electron withdrawing group and the leaving group are optimally selected so bis-alkylation can occur by sequential Michael and retro-Michael reactions. It is also possible to prepare cross-functional alkylating agents with additional multiple bonds conjugated to the double bond or between the leaving group and the electron withdrawing group as described in J. Am. Chem. Soc. 1988, 110, 5211-5212.

A polymer for conjugation in the process of the invention, for example X or X′ in the formulae above, may for example be a polyalkylene glycol, a polyvinylpyrrolidone, a polyacrylate, for example polyacryloyl morpholine, a polymethacrylate, a polyoxazoline, a polyvinylalcohol, a polyacrylamide or polymethacrylamide, for example polycarboxymethacrylamide, or a HPMA copolymer. Additionally the polymer may be one that is susceptible to enzymatic or hydrolytic degradation. Such polymers, for example, include polyesters, polyacetals, poly(ortho esters), polycarbonates, poly(imino carbonates), and polyamides, such as poly(amino acids). The polymer may be a homopolymer, random copolymer or a structurally defined copolymer such as a block copolymer. For example it may be a block copolymer derived from two or more alkylene oxides, or from poly(alkylene oxide) and either a polyester, polyacetal, poly(ortho ester), or a poly(amino acid). Polyfunctional polymers that may be used include copolymers of divinylether-maleic anhydride and styrene-maleic anhydride.

Naturally occurring polymers may also be used, for example polysaccharides such as chitin, dextran, dextrin, chitosan, starch, cellulose, glycogen, poly(sialylic acid) and derivatives thereof. A protein may be used as the polymer. This allows conjugation of one protein, for example an antibody or antibody fragment, to a second protein, for example an enzyme or other active protein. Also, if a peptide containing a catalytic sequence is used, for example an O-glycan acceptor site for glycosyltransferase, it allows the incorporation of a substrate or a target for subsequent enzymatic reaction. Poly(amino acid)s such as polyglutamic acid or polyglycine may also be used, as may hybrid polymers derived from natural monomers such as saccharides or amino acids and synthetic monomers such as ethylene oxide or methacrylic acid.

If the polymer is a polyalkylene glycol, this is preferably one containing C₂ and/or C₃ units, and is especially a polyethylene glycol. A polymer, particularly a polyalkylene glycol, may contain a single linear chain, or it may have branched morphology composed of many chains either small or large. The so-called Pluronics are an important class of PEG block copolymers. These are derived from ethylene oxide and propylene oxide blocks. Substituted polyalkylene glycols, for example methoxypolyethylene glycol, may be used. In a preferred embodiment of the invention, a single-chain polyethylene glycol is initiated by a suitable group, for example an alkoxy, e.g. methoxy, aryloxy, carboxy or hydroxyl group, and is connected at the other end of the chain to the linker group Q.

The polymer may optionally be derivatised or functionalised in any desired way. Reactive groups may be linked at the polymer terminus or end group, or along the polymer chain through pendent linkers; in such case, the polymer is for example a polyacrylamide, polymethacrylamide, polyacrylate, polymethacrylate, or a maleic anhydride copolymer. Such functionalised polymers provide a further opportunity for preparing multimeric conjugates (i.e. conjugates in which the polymer is conjugated to more than one molecule). If desired, the polymer may be coupled to a solid support using conventional methods.

The polymer is suitably hydrophilic or water-soluble, and is preferably a synthetic polymer. The use of polyethylene glycol is particularly preferred.

The optimum molecular weight of the polymer will of course depend upon the intended application. Preferably, the number average molecular weight is in the range of from 500 g/mole to around 75,000 g/mole. When the conjugate is intended to leave the circulation and penetrate tissue, for example for use in the treatment of inflammation caused by malignancy, infection or autoimmune disease, or by trauma, it may be advantageous to use a lower molecular weight polymer in the range 2000-30,000 g/mole. For applications where the conjugate is intended to remain in circulation it may be advantageous to use a higher molecular weight polymer, for example in the range of 20,000-75,000 g/mole. For example, a total polymer molecular weight of 40,000 g/mol can be achieved by using a straight chain 40,000 g/mol polymer, a branched chain polymer having about 20,000 g/mol in each branch, or two separate polymers each of about 20,000 g/mol.

A linking group Q may for example be a direct bond, an alkylene group (preferably a C₁₋₁₀ alkylene group), or an optionally-substituted aryl or heteroaryl group, any of which may be terminated or interrupted by one or more oxygen atoms, sulfur atoms, —NR groups (in which R represents a hydrogen atom or an alkyl (preferably C₁₋₆alkyl), aryl (preferably phenyl), or alkyl-aryl (preferably C₁₋₆alkyl-phenyl) group), keto groups, —O—CO— groups, —CO—O— groups, —O—CO—O, —O—CO—NR—, —NR—CO—O—, —CO—NR— and/or —NR.CO— groups. Such aryl and heteroaryl groups Q form one preferred embodiment of the invention. Suitable aryl groups include phenyl and naphthyl groups, while suitable heteroaryl groups include pyridine, pyrrole, furan, pyran, imidazole, pyrazole, oxazole, pyridazine, primidine and purine. Especially suitable linking groups Q are heteroaryl or, especially, aryl groups, especially phenyl groups, terminated adjacent the polymer X by an —NR.CO— group. The linkage to the polymer may be by way of a hydrolytically labile bond, or by a non-labile bond.

W may for example represent a keto group CO, an ester group —O—CO— or a sulfone group —SO₂—; or, if X-Q-W— together represent an electron withdrawing group, this group may for example be a cyano group.

Substituents which may be present on an optionally substituted aryl or heteroaryl group include for example one or more of the same or different substituents selected from alkyl (preferably C₁₋₄alkyl, especially methyl, optionally substituted by OH or CO₂H), —CN, —NO₂, —CO₂R, —COH, —CH₂OH, —COR, —OR, —OCOR, —OCO₂R, —SR, —SOR, —SO₂R, —NHCOR, —NRCOR, NHCO₂R, —NR.CO₂R, —NO, —NHOH, —NR.OH, —C═N—NHCOR, —C═N—NR.COR, —N⁺R₃, —N⁺H₃, —N⁺HR₂, —N⁺H₂R, halogen, for example fluorine or chlorine, —C≡CR, —C═CR₂ and —C═CHR, in which each R independently represents a hydrogen atom or an alkyl (preferably C₁₋₆alkyl), aryl (preferably phenyl), or alkyl-aryl (preferably C₁₋₆alkyl-phenyl) group. The presence of electron withdrawing substituents is especially preferred. Preferred substituents include for example CN, NO₂, —OR, —OCOR, —SR, —NHCOR, —NR.COR, —NHOH and —NR.COR.

Typical structures in which W and X′ together form a ring include

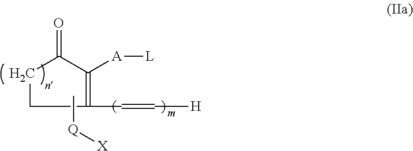

in which n′ is an integer from 1 to 4, and

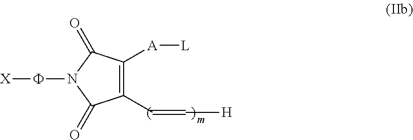

A leaving group L may for example represent —SR, —SO₂R, —OSO₂R, —N⁺R₃, —N⁺HR₂, —N⁺H₂R, halogen, or —OØ, in which R has the meaning given above, and Ø represents a substituted aryl, especially phenyl, group, containing at least one electron withdrawing substituent, for example —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺HR₂, —N⁺H₂R, halogen, especially chlorine or, especially, fluorine, —C≡CR, —C═CR₂ and —C═CHR, in which each R independently has one of the meanings given above.

In a further embodiment of the invention, the protein to be conjugated may be one containing a polyhistidine tag, or “his-tag”, as described in WO 2009/047500. The polymeric conjugating reagents of the formulae (I), (II) and (III) have been found to be useful for conjugation via the polyhistidine tag. Thus a suitable protein, for example INF-β, may be conjugated in the presence of a amphipathic sugar polymer either via a polyhistidine tag or via a reduced disulfide bond.

Throughout this specification and claims, the term “protein” will be used to include proteins and peptides. Suitable proteins which may be conjugated using the process of the invention include for example peptides, polypeptides, antibodies, antibody fragments, enzymes, cytokines, chemokines, receptors, blood factors, peptide hormones, toxin, transcription proteins, or multimeric proteins.

The following gives some specific proteins which may be conjugated using the present invention. Enzymes include carbohydrate-specific enzymes, proteolytic enzymes and the like, for example the oxidoreductases, transferases, hydrolases, lyases, isomerases and ligases disclosed by U.S. Pat. No. 4,179,337. Specific enzymes of interest include asparaginase, arginase, adenosine deaminase, superoxide dismutase, catalase, chymotrypsin, lipase, uricase, bilirubin oxidase, glucose oxidase, glucuronidase, galactosidase, glucocerbrosidase, glucuronidase, and glutaminase.

Blood proteins include albumin, transferrin, Factor VII, Factor VIII or Factor IX, von Willebrand factor, insulin, ACTH, glucagen, somatostatin, somatotropins, thymosin, parathyroid hormone, pigmentary hormones, somatomedins, erythropoietin, luteinizing hormone, hypothalamic releasing factors, antidiuretic hormones, prolactin, interleukins, interferons, colony stimulating factors, hemoglobin, cytokines, antibodies, antibody fragments, chorionicgonadotropin, follicle-stimulating hormone, thyroid stimulating hormone and tissue plasminogen activator.

Certain of the above proteins such as the interleukins, interferons and colony stimulating factors also exist in non-glycosylated form, usually the result of preparation by recombinant protein techniques. It is a significant advantage of the process of the present invention that it enables the conjugation of non-glycosylated versions of polymers, which are generally prone to aggregation and difficult to conjugate using known techniques.

Other proteins of interest are allergen proteins disclosed by Dreborg et al Crit. Rev. Therap. Drug Carrier Syst. (1990) δ 315-365 as having reduced allergenicity when conjugated with a polymer such as poly(alkylene oxide) and consequently are suitable for use as tolerance inducers. Among the allergens disclosed are Ragweed antigen E, honeybee venom, mite allergen and the like.

Glycopolypeptides such as immunoglobulins, ovalbumin, lipase, glucocerebrosidase, lectins, tissue plasminogen activator and glycosilated interleukins, interferons and colony stimulating factors are of interest, as are immunoglobulins such as IgG, IgE, IgM, IgA, IgD and fragments thereof.

Of particular interest are receptor and ligand binding proteins and antibodies and antibody fragments which are used in clinical medicine for diagnostic and therapeutic purposes. The antibody may be used alone or may be covalently conjugated (“loaded”) with another atom or molecule such as a radioisotope or a cytotoxic/antiinfective drug. Epitopes may be used for vaccination to produce an immunogenic polymer—protein conjugate.

Preferably the protein is one which contains a disulfide bond, for example Factor VIIa, Factor VIII, Factor IX, EPO, G-CSF, or, especially, INF-β.

The protein may be derivatised or functionalised if desired. In particular, prior to conjugation, the protein, for example a native protein, may have been reacted with various blocking groups to protect sensitive groups thereon; or it may have been previously conjugated with one or more polymers or other molecules, either using the process of this invention or using an alternative process. It may contain a polyhistidine tag.

The invention further provides a conjugate prepared by the process of the invention per se. The invention further provides a pharmaceutical composition comprising such a conjugate together with a pharmaceutically acceptable carrier; such a conjugate for use as a medicament, especially, where the protein is INF-β, as a medicament for the treatment of multiple sclerosis; and a method of treating a patient which comprises administering a pharmaceutically-effective amount of such a conjugate or pharmaceutical composition to a patient. The invention further provides the use of an amphipathic sugar polymer as a processing aid in a process for the preparation of a conjugate of a polymer and a protein which comprises reacting a polymeric conjugating agent with said protein in an aqueous medium.

The accompanying drawings illustrate results obtained in the following Examples:

FIG. 1 illustrates the result of PEGylation of INF-β in the presence of sugar polymer NV10, Example 1;

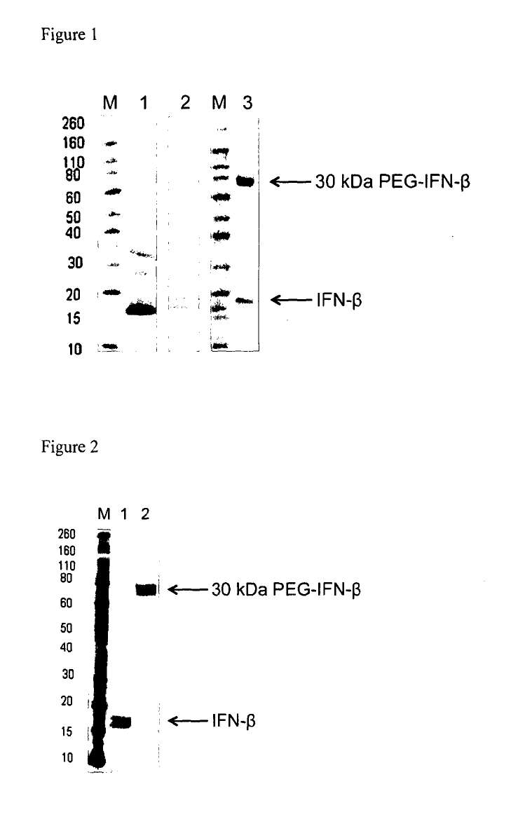

FIG. 2 illustrates the result of the same experiment following further purification of the PEGylated product and silver staining, Example 1;

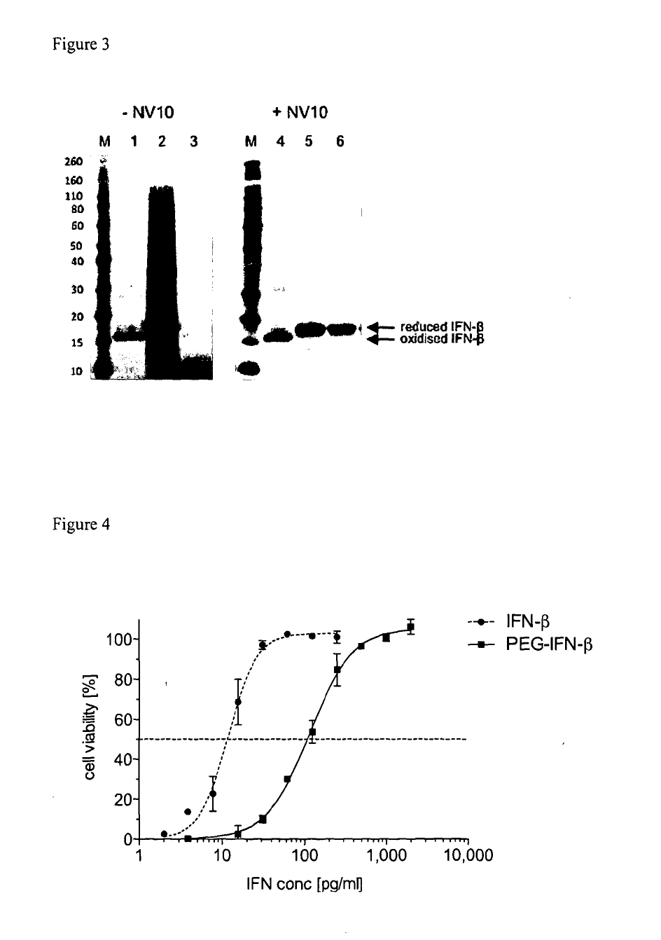

FIG. 3 illustrates the result of reduction of INF-β in the presence and absence of NV10, Example 1;

FIG. 4 illustrates the antiviral activities of native and PEGylated INF-β, Example 1;

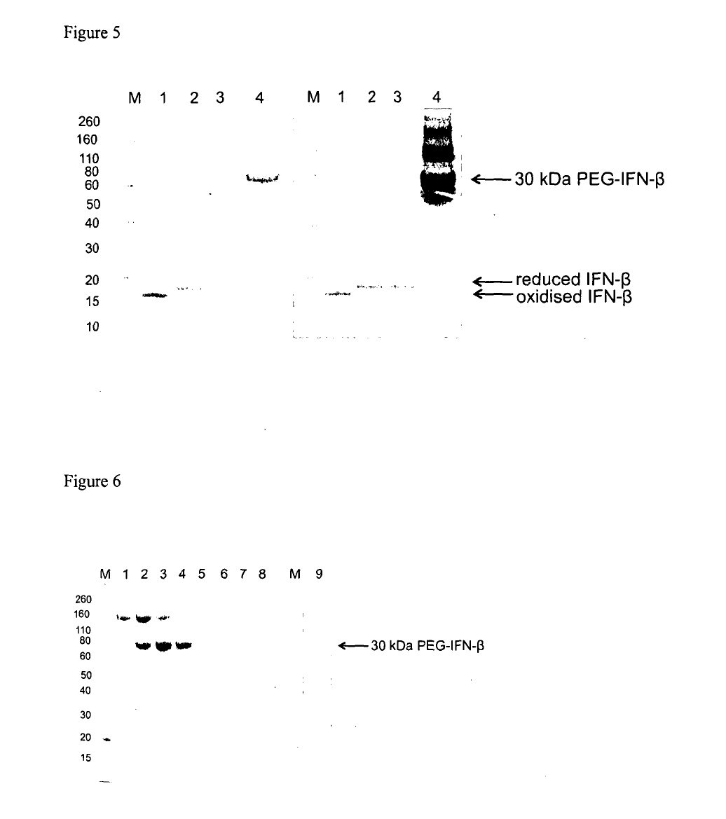

FIG. 5 illustrates the result of PEGylation of INF-13 in the presence of sugar polymer NV10, Example 2;

FIG. 6 illustrates the result of the Example 2 following further purification of the PEGylated product.

The following Examples illustrate the invention.

### EXAMPLE 1

### PEGylation of IFN-β in the presence of NV10 IFN-β (0.5 mg) in 1 ml of 50 mM sodium phosphate, pH 7.8, 0.15 M NaCl, 20 mM EDTA, and 1 mg NV10, was reduced with 23 mM DTT (dithiothreitol) for 30 min. DTT was then removed by buffer exchange with 50 mM phosphate buffer, pH 7.3, 0.15 M NaCl, 20 mM EDTA containing 1 mg/ml sugar polymer NV10 (Trade Mark, available from Expedeon). 5 molar equivalents of 30 kDa PEGylated 4-[2,2-bis[(p-tolylsulfonyl)methyl]acetyl]-benzoic acid PEG reagent of formula

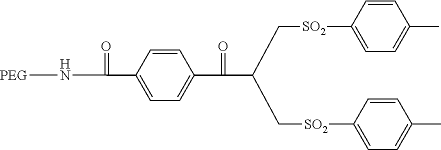

was then added and the reaction was left overnight at 4° C. The reaction mixture was then diluted to 10 ml in 20 mM sodium acetate, pH 5.0 and loaded on an SP FF column pre-equilibrated with the same buffer in order to remove the unreacted PEG reagent. Both native and PEGylated proteins were bound to the ion exchange column and then eluted with an NaCl gradient. To confirm the presence of PEGylated IFN-β, elution fractions were run on SDS-PAGE and the gel was stained first with InstantBlue stain (Expedeon Limited, cat. no. ISBIL) then with barium iodide stain for PEG visualisation.

The results are shown in FIG. 1, in which Lane M is Novex Sharp protein marker; lane 1, IFN-β; lane 2, 30 kda PEGylated IFN-β reaction mixture (InstantBlue stain); lane 3, 30 kDa PEGylated IFN-β eluted from the ion exchange chromatography (barium iodide PEG stain).

Further purification of the PEGylated protein was achieved using size exclusion chromatography. Elution fractions from ion exchange chromatography containing the majority of the PEGylated protein were combined and 200 μl of the pooled sample was loaded on a size exclusion column (Superdex 200, 10/300 GL; GE Healthcare) and eluted in 50 mM sodium phosphate, pH 7.5, 0.15 NaCl, 10 mM EDTA. The purity of the PEGylated IFN-β was confirmed by silver stain.

The results are shown in FIG. 2, in which Lane M is Novex Sharp marker; lane 1, eluted IFN-β; lane 2, purified 30 kDa PEGylated IFN-β.

The effect of NV-10 on IFN-β aggregation during protein reduction was investigated in the following experiment. 100 μl of IFN-β (0.5 mg/ml in 50 mM phosphate, pH 7.8) was reduced for 1 h with 30 mM DTT in the presence and absence of 1 mg/ml sugar polymer NV-10 (Trade Mark; Expedeon Limited). For the sample without NV-10, DTT was removed using a NAP-5 desalting column pre-equilibrated with 50 mM sodium phosphate, pH 7.4. For the sample containing NV-10, DTT removal was performed using a NAP-5 desalting column pre-equilibrated with 1 mg/ml of NV10 in 50 mM sodium phosphate, pH 7.4. Protein contents were analysed by SDS-PAGE, shown in FIG. 3, in which the first SDS-PAGE shows the results without NV10, and the second shows the results with NV10.

IFN-β that was reduced in 30 mM DTT in the presence of 1 mg/ml NV10 allowed significant recovery of reduced monomeric protein following a buffer exchange step to remove DTT, whereas recovery of reduced protein was poor as the aggregated protein which was formed (and which is seen as an extensive smear on the SDS-PAGE) did not pass through the column. Lane M is Novex Sharp marker; lanes 1 and 4, IFN-β; lane 2, reduced IFN-β in 30 mM DTT; lane 3, elution fraction from NAP-5 column; lane 5, reduced IFN-β in 30 mM DTT in the presence of 1 mg/ml NV-10; lane 6, elution fraction from NAP-5 desalting column pre-equilibrated with NV-10.

In Vitro Biological Evaluation of PEGylated IFN-β

The activity of the 30 kDa PEGylated IFN-β was evaluated using an antiviral assay. A549 cells were resuspended in DMEM/10% FCS supplemented with penicillin and streptomycin at a concentration of 0.2×10⁶ cells/ml and 50 μl of the cells were added to each well in a 96-well flat-bottom microtiter plate (Nunc). Cells were then allowed to adhere overnight at 37° C. On the following day, PEGylated and native IFN-β were prepared in a 2-fold dilutional series, and 50 μl of each dilution was added to the wells. Each protein sample was tested in quadruplicate. The plate was then incubated for 24 h. On the following day, the medium was removed and the cells were inoculated with EMCV working solution prepared in DMEM/10% FCS (50 μl/well). Cells were left for further 24 h. On the following day, wells were washed with 300 μl/well of PBS and the remaining adherent cells were stained with 4% formaldehyde/0.1% methyl violet (50 μl/well) for 30 min. The plate was then washed twice with 300 of PBS and air-dried. The dye was solubilised with 2% SDS solution (50 μl/well) and the absorbance was measured at 570 nm. The results are shown in FIG. 4, from which it can be seen that antiviral activity was retained. The in vitro antiviral activity of the native IFN-β showed an ED₅₀ of 11±2 pg/ml (n=3). 30 kDa PEG-IFN-β showed an ED₅₀ of 93±10 pg/ml (n=4). This represents 12% activity compared to IFN-β.

### EXAMPLE 2

### PEGylation of IFN-β in the Presence of NV10

IFN-β (0.5 mg) in 1 ml of 50 mM sodium phosphate, pH 7.8, 0.15 M NaCl, 10 mM EDTA, 2 mg/ml NV-10, was reduced with 30 mM DTT for 45 min. DTT was then removed by buffer exchange using a PD-10 desalting column (GE Healthcare, cat. no. 17-0851-01) pre-equilibrated with 50 mM phosphate buffer, pH 7.3, 0.15 M NaCl, 20 mM EDTA containing 1 mg/ml NV10. 3 molar equivalents of the 30 kDa PEG reagent of Example 1 was then added and the reaction was left overnight at 4° C. The presence of PEGylated protein was confirmed first by InstantBlue stain for protein visualisation and then with barium iodide staining for PEG visualisation. The results are shown in FIG. 5, in which the left gel shows InstantBlue staining, and the right, InstantBlue/barium iodide PEG stain. Lane M is Novex Sharp protein marker; lane 1, IFN-β; lane 2, reduced IFN-β in the presence of NV-10 and DTT; lane 3, reduced IFN-β in the presence of NV-10 after DTT removal; lane 4, 30 kDa PEGylation reaction mixture.

Ion exchange and size exclusion chromatography were performed to obtain purified PEGylated IFN-β. The reaction mixture was buffer exchanged on PD-10 desalting column for 20 mM sodium acetate, pH 5.5 and loaded on an SP HP column pre-equilibrated with the same buffer. After washing in 20 mM sodium acetate, pH 5.5 in order to remove the unreacted PEG reagent, a NaCl gradient elution was performed using 20 mM sodium acetate, pH 5.5, 0.8 M NaCl. Further purification of the PEGylated protein was achieved using size exclusion chromatography. Elution fractions from ion exchange chromatography containing the majority of the PEGylated protein were combined. 200 μl of the pooled sample was loaded on a size exclusion column (Superdex 200, 10/300 GL; GE Healthcare) and eluted in 50 mM sodium phosphate, pH 7.5, 0.15 NaCl. Purity of the PEGylated IFN-β was confirmed by SDS-PAGE analysis.

The results are shown in FIG. 6, which shows SDS-PAGE of the fractions eluted from ion exchange chromatography (Lanes 1-8) and size exclusion chromatography (lane 9). Lane M is Novex Sharp marker; lanes 1-8, Fractions from a NaCl gradient elution from SP HF; lane 9, purified 30 kDa PEGylated IFN-β. PEG stain was used to confirm the presence of PEGylated protein.

The in vitro antiviral activity of the native IFN-β tested as in Example 1 showed an ED₅₀ of 11±2 pg/ml (n=3). 30 kDa PEG-IFN-β showed an ED₅₀ of 93±10 pg/ml (n=4). This represents a 12% activity compared to IFN-β.

1. A process for the conjugation of a polymer to a protein, which comprises reacting a polymeric conjugating agent with said protein, in an aqueous medium, in the presence of an amphipathic sugar polymer.

2. A process as claimed in claim 1, in which the sugar polymer contains non-polar, hydrophobic substituents selected from the group consisting of alkyl groups having from 1 to 25 carbon atoms, alkenyl and alkynyl groups having from 2 to 25 carbon atoms, haloalkyl groups having from 1 to 25 carbon atoms, cycloalkyl groups having from 3 to 9 carbon atoms, aryl groups having from 6 to 14 carbon atoms, aralkyl groups comprising alkyl groups having from 1 to 25 carbon atoms which are substituted with 1 or more aryl groups having from 6 to 14 carbon atoms, fatty acid groups having from 2 to 25 carbon atoms and polyols having from 1 to 25 carbon atoms.

3. A process as claimed in either claim 1, in which the sugar polymer is a compound of the general formula
G(O—CO—NH—R¹)ₐ—[F(O—CO—NH—R²)_{b}]ₙ (IV)
wherein:
G is a terminal glucosyl unit in which one or more hydroxyl groups thereof may be substituted with a group or groups of formula (O—CO—NH—R¹);
R¹ is a hydrocarbyl group selected from the group consisting of alkyl groups, alkenyl groups, alkynyl groups, haloalkyl groups, cycloalkyl groups, aryl groups and aralkyl groups and, where there is more than one (O—CO—NH—R¹) group on the glucosyl unit, each R¹ group may be the same or different;
a is an integer of from 0 to 4;
F is a fructosyl unit in which one or more hydroxyl groups thereof may be substituted with a group or groups of formula (O—CO—NH—R²);
R² is a hydrocarbyl group selected from the group consisting of alkyl groups, alkenyl groups, alkynyl groups, haloalkyl groups, cycloalkyl groups, aryl groups and aralkyl groups and, where there is more than one (O—CO—NH—R²) group on the fructosyl unit, each R² group may be the same or different; b is an integer of from 0 to 3 and from 0 to 4 for the terminal fructosyl unit;
n is an integer of from 2 to 499 preferably of from 2 to 249, 2 to 99, 2 to 49, 9 to 49, 14 to 39, 19 to 29, or 19 to 24,
each unit of formula F(O—CO—N H—R²)_{b} may be the same or different from any other unit of formula F(O—CO—N H—R²)_{b}; and
the average degree of substitution per glucosyl or fructosyl unit is from 0.01 to 3.0.

4. A process as claimed in claim 1, in which the sugar polymer is an uncharged polyfructose derivative of molecular weight about 5 kDa having a linear carbohydrate backbone, and hydrophobic modifications.

5. A process as claimed in claim 1, in which the protein to be conjugated is of low solubility or which tends to aggregate in an aqueous medium, or is a protein which becomes of lower solubility or which shows an increased tendency to aggregate during the course of a conjugation reaction in an aqueous medium in the absence of a sugar polymer.

6. A process as claimed in claim 1, in which the protein to be conjugated is Factor VIIa, Factor VIII, Factor IX, EPO, G-CSF, or INF-β.

7. A process as claimed in claim 1, in which the polymer to be conjugated to the protein is a homopolymer or copolymer of polyalkylene glycol, polyvinylpyrrolidone, polyacrylate, polymethacrylate, polyoxazoline, polyvinylalcohol, polyacrylamide, polymethacrylamide, HPMA copolymer, polyester, polyacetal, poly(ortho ester), polycarbonate, poly(imino carbonate), polyamide, copolymer of divinylether-maleic anhydride and styrene-maleic anhydride, polysaccharide, or polyglutamic acid.

8. A process as claimed in claim 7, in which said polymer is polyethylene glycol.

9. A process as claimed in claim 1, in which the polymeric conjugation reagent is one which conjugates to thiol groups.

10. A process as claimed in claim 1, which is a process for the conjugation of a polymer to a protein containing a disulfide bond, which comprises reducing said disulfide bond in an aqueous medium in the presence of an amphipathic sugar polymer, and subsequently reacting the reduced product with a polymeric conjugating agent.

11. A process as claimed in claim 10, in which said polymeric conjugation agent is either (i) a compound of the general formula 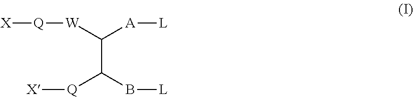
in which one of X and X′ represents a polymer and the other represents a hydrogen atom;
Q represents a linking group;
W represents an electron-withdrawing group, for example a keto group, an ester group —O—CO— or a sulfone group —SO₂—; or, if X′ represents a polymer, X-Q-W together may represent an electron withdrawing group;
A represents a C₁₋₅ alkylene or alkenylene chain;
B represents a bond or a C₁₋₄ alkylene or alkenylene chain; and
each L independently represents a leaving group;
or (ii) a compound of the general formula 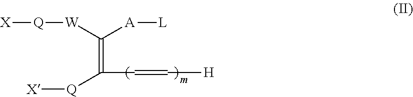
in which X, X′, Q, W, A and L have the meanings given for the general formula I, and in addition if X represents a polymer, X′ and electron-withdrawing group W together with the interjacent atoms may form a ring, and m represents an integer 1 to 4;
or (iii) a compound of the general formula
X-Q-W—CR³═C4²-L (III)
in which X, Q and W have the meanings given for the general formula I, and either R³ represents a hydrogen atom or a C₁₋₄alkyl group and L represents a leaving group, or R³ and L together represent a bond; and R⁴ represents a hydrogen atom or a C₁₋₄ alkyl group.

12. A process as claimed in claim 11, which comprises the additional step of reducing the electron withdrawing group W after conjugation has taken place.

13. A process as claimed in claim 11, in which Q represents a direct bond, an alkylene group, or an optionally-substituted aryl or heteroaryl group, any of which may be terminated or interrupted by one or more oxygen atoms, sulfur atoms, —NR groups (in which R represents a hydrogen atom or an alkyl, aryl, or alkyl-aryl group), keto groups, —O—CO— groups, —CO—O— groups, —O—CO—O, —O—CO—NR—, —NR—CO—O—, —CO—NR— and/or —NR.CO— groups.

14. A process as claimed in claim 11, in which W represents a keto group CO, an ester group —O—CO— or a sulfone group —SO₂—, or, if X-Q-W— together represent an electron withdrawing group, a cyano group.

15. A process as claimed in claim 11, in which a leaving group L represents —SR, —SO₂R, —OSO₂R, —N⁺R₃,
—N⁺HR₂, —N⁺H₂R, halogen, or —OØ, in which R represents a hydrogen atom or an alkyl, aryl, or alkyl-aryl group, and Ø represents a substituted aryl group containing at least one electron withdrawing substituent.

16. A conjugate of a protein with a polymer prepared by the process of the invention.

17. A pharmaceutical composition comprising a conjugate as claimed in claim 16, together with a pharmaceutically acceptable carrier.

18. (canceled)

19. A method of treating a patient which comprises administering a pharmaceutically-effective amount of a conjugate as claimed in claim 16 to a patient.

20. (canceled)

21. A process as claimed in claim 1, in which the sugar polymer is an uncharged polyfructose derivative of molecular weight about 5 kDa having a linear carbohydrate backbone, and hydrophobic modifications, and in which the protein to be conjugated is Factor VIIa, Factor VIII, Factor IX, EPO, G-CSF, or INF-β.
